(19) United States
(12) Patent Application Publication
(10) Pub. No.: US 2021/0369896 A1
(43) Pub. Date: Dec. 2, 2021

(54) Cold Plasma Sanitization Robot

(71) Applicant: Nanoclean (S) Pte Ltd, SG (SG)

(72) Inventors: Thomas Angelo Steiber, San Jose, CA (US); Chen Sau Ngen, SG (SG); Chen Nam Hin, SG (SG); Nikolaos Giannaris, San Carlos, CA (US)

(21) Appl. No.: 17/336,289

(22) Filed: Jun. 1, 2021

Related U.S. Application Data
(60) Provisional application No. 63/033,071, filed on Jun. 1, 2020.

(51) Int. Cl. A61L 2/14; A61L 9/22; A61L 2/26

(52) U.S. Cl. CPC A61L 2/14; A61L 9/22; A61L 2202/25; A61L 2202/26; A61L 2/26

(57) ABSTRACT

A cold plasma system to disinfect PPE while disinfecting a floor surface, comprising a positive air ion collector on a top portion and a negative air ion collector on a lower portion.

### CROSS-REFERENCE TO RELATED APPLICATIONS

This application claims priority under 35 USC 1219(e) to U.S. Prov. App. No. 63/033,071, filed Jun. 1, 2020, the content of which is being hereby incorporated by reference in its entirety.

### FIELD OF THE INVENTION

The invention relates generally to an electronic sanitization, and more specifically, to a mobile robot in a cold plasma system to neutralize particles with negative ion emissions and to capture the neutralized particles with positively charged collectors.

### BACKGROUND

With the recent outbreak of global pandemics such as the novel COVID-19 virus from the Coronavirus family, environmental disinfection of densely populated indoor areas is of critical importance. These viruses and bacteria need to be removed from surfaces as well as the air.

Problematically, conventional techniques for disinfection typically require manual application. Furthermore, manual or even automated disinfection is typically performed overnight or when a space has low traffic. Many techniques leave the area wet and consequently out of commission until dry. This makes disinfection difficult in airports or trains stations that operate 24-hours a day leaving small windows for cleaning without disrupting foot traffic.

Therefore, what is needed is a cold plasma system using a mobile robot for disinfecting and collecting in indoor environments.

### SUMMARY

To address the problems of the prior art, devices, methods, and computer-readable medium are provided for a cold plasma system for disinfection and collection in indoor environments, are disclosed. For example, a mobile robot with self-navigation and object sensing can keep indoor areas safe from COVID-19 and other health threats.

In one embodiment, a cold plasma system drastically reduces airborne diseases caused by pathogenic microbes small enough to be discharged from an infected person, thus reducing the transmission of diseases. One implementation automatically moves around an area in a predetermined pattern to ensure adequate coverage. Sensors can avoid people, furniture and walls. Another implementation includes a cavity for quick disinfection of masks, gloves, hands, and the like. Still another implementation is manually controlled with a remote control.

In a specific embodiment, the cold plasma system includes a mobile robot with a collector head connected by a vertical neck to a base with a negative ion emitter. The collector head can be positively charged +15 k volt DC collection plates, as well as three additional positively charged +15 k volt DC collection plates under the robot base in order to provide disinfection activity at all times (see FIG. 7). One implementation includes an ionizing assisted fan on the negatively charged −15 k volt DC ion emitter. In an implantation, the mobile robot includes sensors, processing, and location technologies to dynamically guide the process in a space with lots of traffic.

Advantageously, particles, virus, and other contaminants are reduced with minimal disruption.

### BRIEF DESCRIPTION OF THE DRAWINGS

Embodiments will now be described by way of examples, with reference to the accompanying drawings which are meant to be exemplary and not limiting. For all figures mentioned herein, like numbered elements refer to like elements throughout.

FIG. 1 illustrates several perspective views of a mobile robot in a cold plasma system for disinfecting and collecting in indoor environments, according to an embodiment.

FIG. 2 illustrates the cold plasma system responding to airborne particles, according to some embodiments.

FIG. 3 illustrates disinfection of Agent Orange, and other threats, collected by the cold plasma system, according to one embodiment.

FIG. 4 illustrates relative size of airborne contaminants collected by the cold plasma system, according to an embodiment.

FIG. 5 illustrates several views a collector head of the cold plasma system, according to an embodiment.

FIG. 6 illustrates a schematic with several views of the collector head, according to an embodiment.

FIG. 7 illustrates positive and negative washable and reusable pads of the collector head, according to an embodiment.

FIG. 8 illustrates a bottom view of negative charge plates and wheels of an emitter base of the cold plasma system, according to an embodiment.

FIGS. 9A & 9B illustrate the cold plasma system installed an airplane cabin, according to an embodiment.

FIGS. 10A & 10B illustrate additional features of the cold plasma system, according to an embodiment.

### DETAILED DESCRIPTION

I. Disinfection and Collection Robot

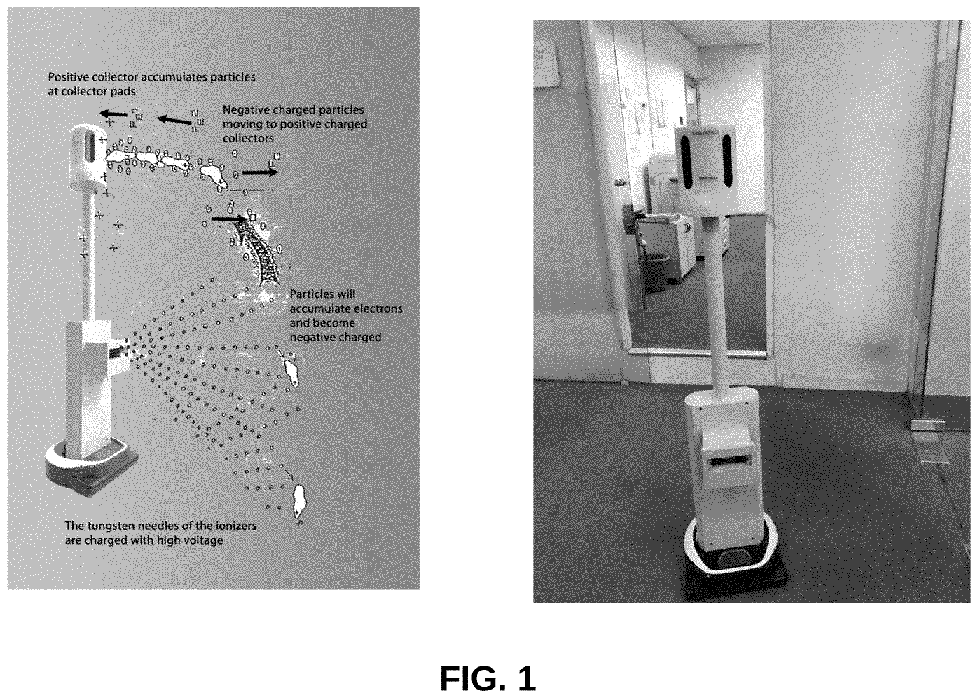

FIG. 1 illustrates several views of a cold plasma system for disinfecting and collecting in indoor environments while in use, according to an embodiment. The cold plasma system in the case of FIG. 1 is a mobile robot, although many other implementations are possible. The mobile robot can roam semiconductor cleanrooms, office environments, large public facilities, industrial areas, and the like.

The mobile robot comprises generally a collector head supported by a neck to an emitter base. The neck can be hollow to enclose data and power cables connecting components of the collector head and the emitter base. The mobile robot can also include computer hardware, computer software, a digital display, a Wi-Fi or Bluetooth communication system, a battery, and other electronic components. The robots may need periodic system tuning and calibration in 6-month intervals, rather than weekly intervals as required by some conventional systems. The electrodes are preferably long lasting.

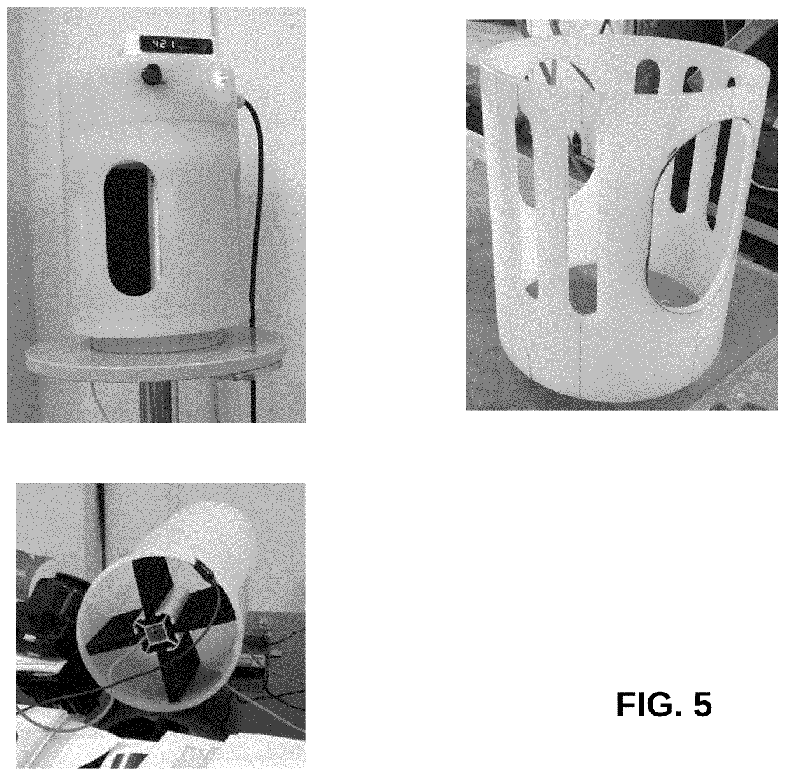

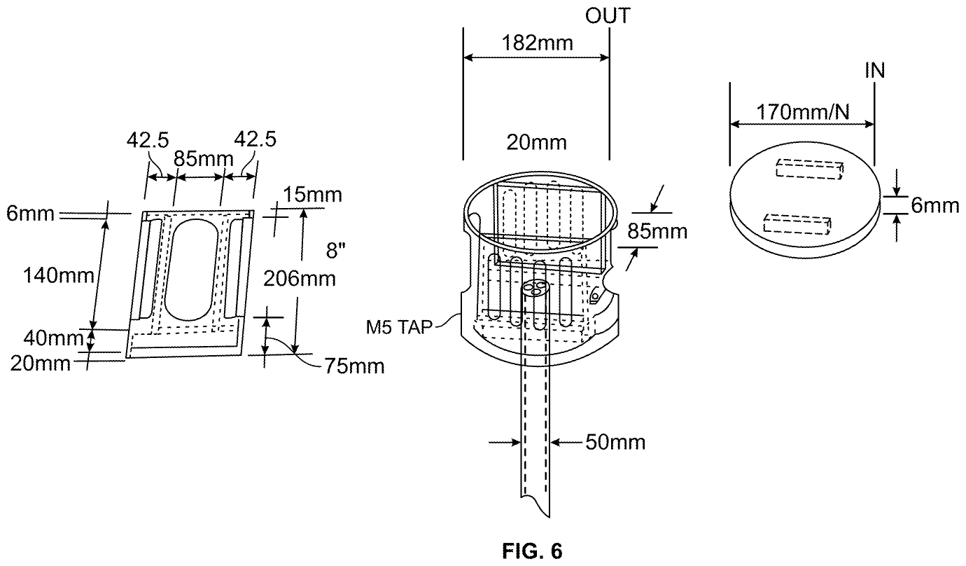

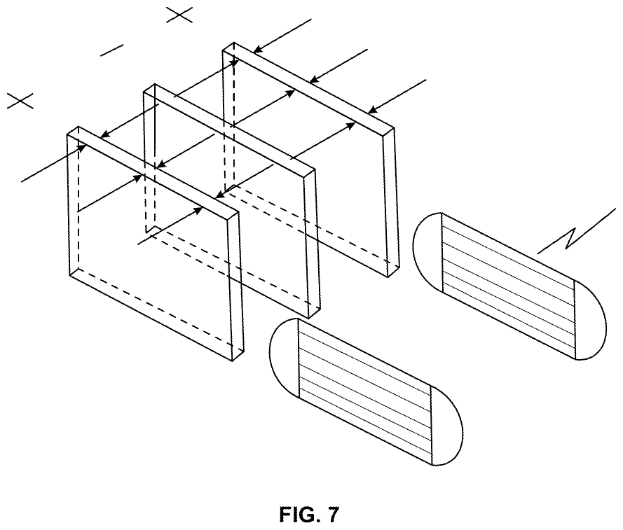

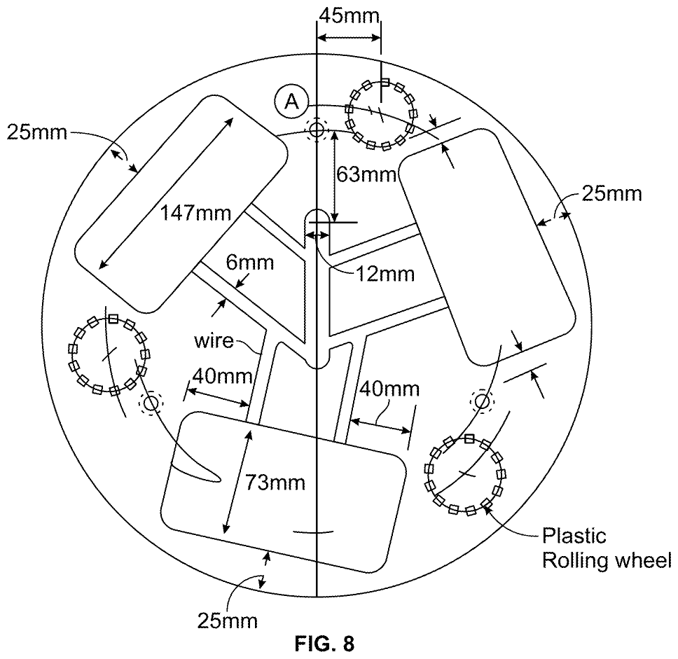

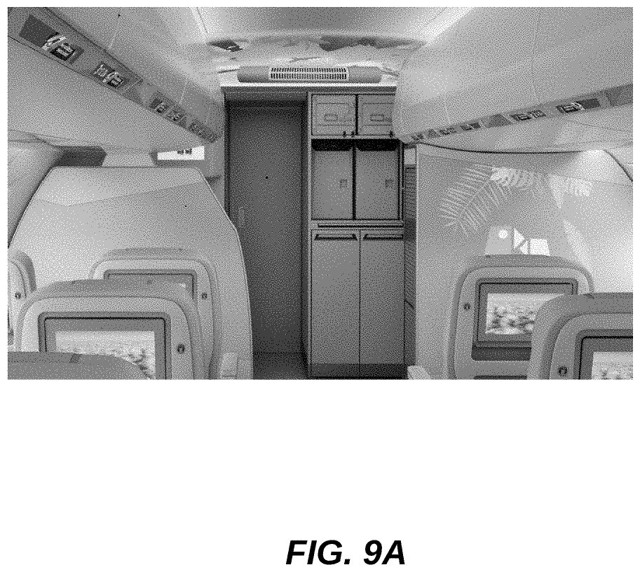

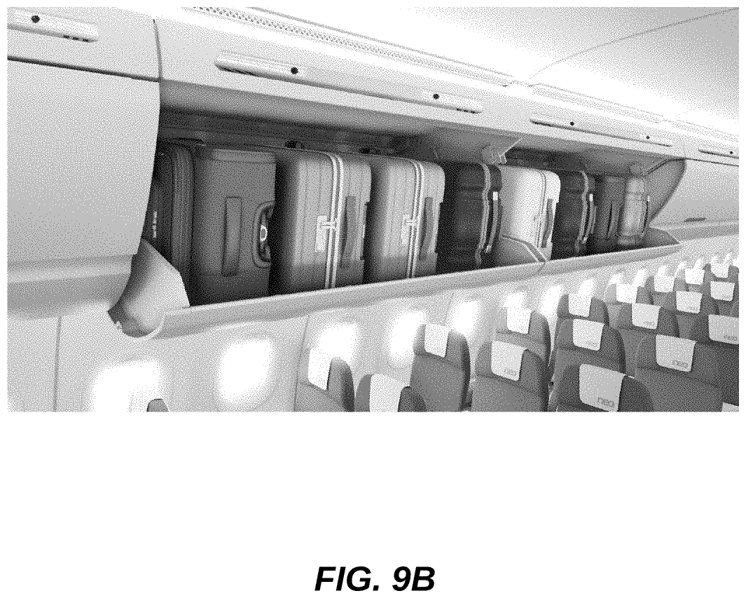

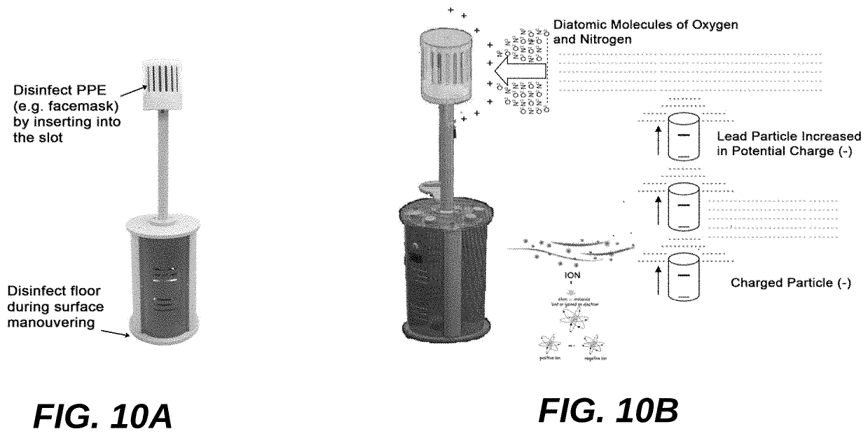

An embodiment of the collector head is shown in FIG. 5. The collector head includes a shell shown in a detail view and collector plates shown in a bottom view. An example detail is shown in FIG. 6 with three collector plates and insulation material. A detailed schematic example is shown with several views in FIG. 6. In one instance, the collector head can be positively charged +15 k volt DC collection plates, as well as three additional positively charged +15 k volt DC collection plates under the robot base in order to provide disinfection activity at all times (see FIG. 8). The foam collection filter media shown in FIG. 7 can be washable and reusable.

A cavity in the collector head can allow for disinfection of inserted objects (e.g., −2 KV to −3 KV). For instance, human hands, an n-95 or surgical mask, gloves, PPE (Personal Protective Equipment), car keys, cell phones, kitchen silverware, or the like can be inserted quickly and conveniently.

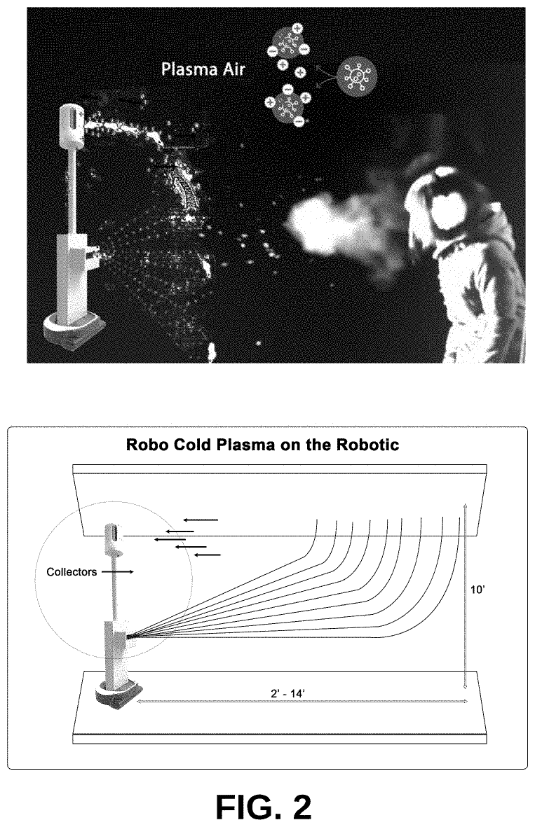

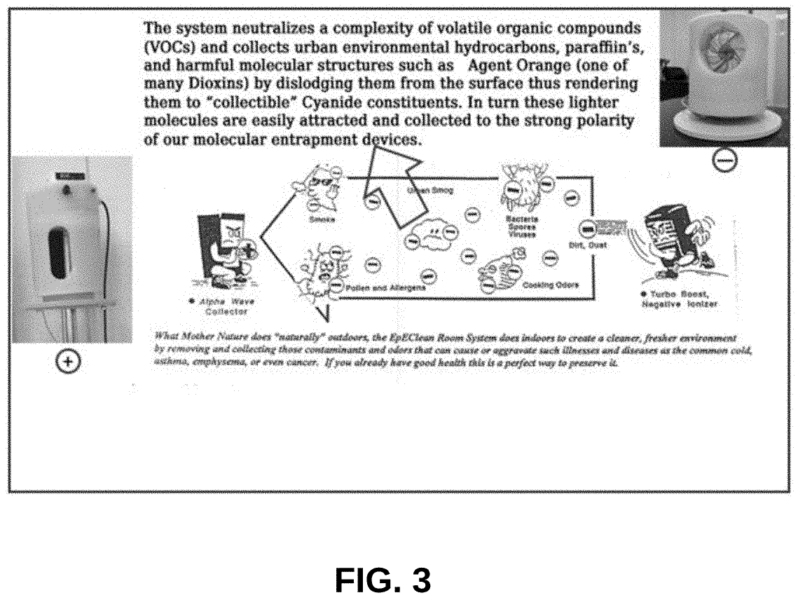

An embodiment of the emitter base is shown in FIG. 8. The tungsten needles of the ionizers are charged with high voltage. The emitter base discharges the negatively charged ions so that particles will accumulate electrons and become negatively charged. Ultimately, the negatively charged particles are captured by the positive charge of the collector head (see FIGS. 2 and 3). One implementation includes an ionizing assisted fan on the negatively charged −15 k volt DC ion emitter. Additionally, the emitter base can include wheels for zero turn mobility around an indoor area.

II. Operation of the Robot

In operation, the cold plasma system provides a uniform ion matrix distribution in a given space which in turn allows for a more efficient collection of micron/sub-micron viral constituents. Using its mobility, the robot of FIG. 1 can sweep an area using a predetermined pattern for efficiency and coverage. Disinfection operations can have stricter parameters for virus disinfection programs or clean rooms versus general cleaning environments. In more detail, when disinfecting for viruses, coverage patterns can be tightened to meet higher standards.

Alternatively, an object sensing robot can self-navigate by adjusting the predetermined pattern based on known or unknown objects. Yet another variation uses intelligent routing with a retrofitted camera and a UV inspection lamp in order to scan the floor cleanliness for live monitoring at all times. Thus, roaming patterns can be adjusted on-the-fly.

One implementation centrally manages a pool of mobile robots in cooperation around a large area for optimal coverage amongst the aggregated area covered by the mobile robots. A centralized controller adjusts individual mobile robot routes based on dynamic feedback.

III. Advantages of the Cold Plasma System

In general, the cold plasma system neutralizes a complexity of volatile organic compounds (VOCs) and collects urban environmental hydrocarbons, paraffins, and harmful molecular structures, such as Agent Orange, as shown in FIGS. 2 and 3. The microorganisms are killed on contact and removed.

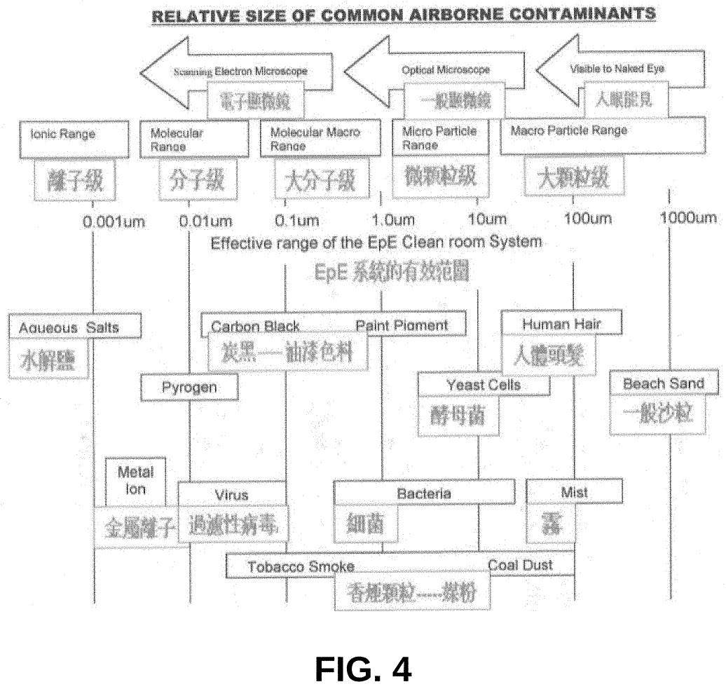

The cold plasma system has been proven to collect particle/species geometries anywhere from 1000 microns to as small as 0.001 microns (10 Angstroms), as verified by Scanning and Transmission Electron Microscopy and other highly accurate analytical instrumentation. A relative size chart of various contaminants are shown in FIG. 4. The cold plasma system guarantees a safer, disease free environment by removing and collecting potentially hazardous, contagious, respirable contaminants down to the Angstrom level.

The cold plasma system is preferably safe and designed to be extremely operator friendly. There are no radio frequency or electromagnetic interferences (RFI/EMI) generated. One embodiment operates only with low voltage, low current (DC) sources. The cold plasma system can be implemented to generate absolutely zero harmful ozone, for high volume class 10 cleanrooms and clinical.

IV. General Computing Devices

Many of the functionalities described herein can be implemented with computer software, computer hardware, or a combination.

Computer software products (e.g., non-transitory computer products storing source code) may be written in any of various suitable programming languages, such as C, C++, C#, Oracle® Java, JavaScript, PHP, Python, Perl, Ruby, AJAX, and Adobe® Flash®. The computer software product may be an independent application with data input and data display modules. Alternatively, the computer software products may be classes that are instantiated as distributed objects. The computer software products may also be component software such as Java Beans (from Sun Microsystems) or Enterprise Java Beans (EJB from Sun Microsystems).

Furthermore, the computer that is running the previously mentioned computer software may be connected to a network and may interface to other computers using this network. The network may be on an intranet or the Internet, among others. The network may be a wired network (e.g., using copper), telephone network, packet network, an optical network (e.g., using optical fiber), or a wireless network, or any combination of these. For example, data and other information may be passed between the computer and components (or steps) of a system of the invention using a wireless network using a protocol such as Wi-Fi (IEEE standards 802.11, 802.11a, 802.11b, 802.11e, 802.11g, 802.11i, 802.11n, and 802.ac, just to name a few examples). For example, signals from a computer may be transferred, at least in part, wirelessly to components or other computers.

In an embodiment, with a Web browser executing on a computer workstation system, a user accesses a system on the World Wide Web (WWW) through a network such as the Internet. The Web browser is used to download web pages or other content in various formats including HTML, XML, text, PDF, and postscript, and may be used to upload information to other parts of the system. The Web browser may use uniform resource identifiers (URLs) to identify resources on the Web and hypertext transfer protocol (HTTP) in transferring files on the Web.

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

We claim:

1. A cold plasma system to reduce pathogenic microbes by disinfecting PPE while disinfecting a floor surface, comprising:
a positive air ion collector on a top portion; and
a negative air ion collector on a lower portion, connected to the top portion by a neck,
wherein the cold plasma system moves around an area in a predetermined pattern.
